(19) United States
(12) Patent Application Publication
(10) Pub. No.: US 2020/0306072 A1
(43) Pub. Date: Oct. 1, 2020

(54) COMFORT HARNESS FOR ORTHOTIC BRACE

(71) Applicant: Orthocare Medical Equipment, LLC, Manchester, NH (US)

(72) Inventors: Rita CARNEY, Auburn, NH (US); Steve SANTANIELLO, Milford, NH (US)

(21) Appl. No.: 16/830,353

(22) Filed: Mar. 26, 2020

Related U.S. Application Data
(60) Provisional application No. 62/823,795, filed on Mar. 26, 2019.

(51) Int. Cl. A61F 5/30; A61F 5/058; A61F 5/01; A61F 7/02

(52) U.S. Cl. CPC A61F 5/30; A61F 7/02; A61F 5/0102; A61F 5/058

(57) ABSTRACT

A comfort harness for an orthotic brace being fully adjustable and having a crescent-shaped pad located about the front shoulder of a wearer for dispersing a load and for added comfort for the wearer. Multiple straps are provided for upper back, under arm, and cross-body support for the wearer. The comfort harness configured to connect with a variety of orthotics for an arm, a shoulder, and the like. In some cases, the comfort harness is capable of being donned and adjusted by the wearer when in a supine position.

### FIELD OF THE DISCLOSURE

The present disclosure relates to orthotic braces and more particularly to a comfort harness for use with orthotic braces.

### BACKGROUND OF THE DISCLOSURE

Conventional orthotics all have a single straight strap or pad that lays directly over the shoulder of a wearer. These pads do not conform to the anatomy of the wearer and typically pull downwards against the body and neck region causing discomfort and pain.

Another prior orthotic has three straps meeting at a frontal pad. However, the three connections are all fixed and non-adjustable, and the pad is square shaped such that the strap connecting down to the front of the orthotic does not allow for rotation. Additionally, all three straps connect to a central ring and apply unwanted pressure against a single point on the body. These straps also move independently on the ring causing excessive movement and rotation. Finally, all three of the intersecting straps meet at a connector ring and the closed loop formed by the support straps is passed around the extended arm towards the patient's torso which creates a major fitting issue. Essentially, this requires the sliding of an arm through the strap assembly as opposed to having the assembly wraps around the wearer from front to back.

While some prior system may alleviate some neck pull, the single strap approach still crosses over the shoulder to a sling in the front. There, a back connector unit has strap running vertical down towards the belt and across to sling. As weight is applied to the single strap over the shoulder the belt wants to rise up on the wearer's back. Force is still only applied to one section of the shoulder and does not distribute the load evenly.

Other systems attempt to alleviate the shifting of the sling unit, but the single strap still goes over the shoulder unit with a second strap going under the arm but coming around to the back portion of the orthotic unit. The single strap does not diffuse the load and does not disperse load on the sling but instead is used to stop rotation of the sling. Regardless, these conventional systems do not have adjustability at the section attached to the shoulder. The three straps are all stitched directly to a single pad. Adjustment happens only at the lower sling and around the back lower sling and pillow. A wearer cannot release away from the shoulder without moving away from a chair or a bed.

As noted previously, the conventional systems have several issues that are addressed with the present disclosure. Some issues are having a single strap over the shoulder without a release capability from the arm in a sitting position. A single pad over the shoulder does not disperse the load across the chest but pulls both straps down to buckle on sling and does not anatomically conform to the shape of the chest and shoulder area. The single strap and pad that runs over the shoulder and still places pressure on the neck area. The second strap connection that runs horizontally around the waist is to facilitate an immobilization of the arm. This design does not dispersing the load across the shoulder area. Although the horizontal waist strap is removable to allow arm (mobilization) the neck strap is fixed and not removable. Access to the shoulder and arm of the carrying side would mean the patient has to slide the arm out. A wearer could not be sitting in a chair or lying in bed and easily remove the brace.

Wherefore it is an object of the present disclosure to overcome the above-mentioned shortcomings and drawbacks associated with the conventional harnesses for orthotic braces. The present disclosure overcomes several problems with conventional orthotics by providing a frontally removable and adjustable harness with a unique crescent shaped pad. The crescent shape pad anatomically contours and disperses the load across the front of the chest and shoulder region.

### SUMMARY OF THE DISCLOSURE

One aspect of the present disclosure is a comfort harness for an orthotic, comprising: a crescent-shaped pad having an upper portion and a lower portion, the upper portion being configured to reversibly attach to an upper back strap and the lower portion being configured to reversibly attach to an under arm strap; a multi-strap connector being configured to reversibly attach to the upper back strap and the under arm strap; and at least one cross-body strap being configured to reversibly attach to an orthotic.

One embodiment of the comfort harness for an orthotic is wherein the at least one cross-body strap comprises a first and a second cross-body strap.

Another embodiment of the comfort harness for an orthotic is wherein the first cross-body strap is reversibly attached to the crescent-shaped pad and reversibly attached to the orthotic.

Yet another embodiment of the comfort harness for an orthotic is wherein the second cross-body strap is permanently attached to the multi-strap connector and reversibly attached to the orthotic. In some cases, the crescent-shaped pad is reinforced. In some embodiments, the crescent-shaped pad further comprises cooling fabric.

In certain embodiment of the comfort harness for an orthotic, the upper back and the under arm straps comprises stretchable fabric. In some cases, the multi-strap connector comprises two or more slots for receiving and orienting straps. In certain embodiments, the multi-strap connector comprises a LDPE foam laminate.

Another aspect of the present disclosure is an adjustable comfort harness for an orthotic, comprising: a crescent-shaped pad having an upper portion and a lower portion, the upper portion being configured to reversibly attach to an upper back strap and the lower portion being configured to reversibly attach to an under arm strap; a multi-strap connector comprising at least two slots for receiving straps, the multi-strap connector being configured to reversibly attach to the upper back strap and the under arm strap; and a first and a second cross-body strap being configured to reversibly attach to an orthotic.

These aspects of the disclosure are not meant to be exclusive and other features, aspects, and advantages of the present disclosure will be readily apparent to those of ordinary skill in the art when read in conjunction with the following description, appended claims, and accompanying drawings.

### BRIEF DESCRIPTION OF THE DRAWINGS

The foregoing and other objects, features, and advantages of the disclosure will be apparent from the following description of particular embodiments of the disclosure, as illustrated in the accompanying drawings in which like reference characters refer to the same parts throughout the different views. The drawings are not necessarily to scale, emphasis instead being placed upon illustrating the principles of the disclosure.

FIG. 1A shows a front perspective view of one embodiment of the comfort harness of the present disclosure.

FIG. 1B shows a back perspective view of one embodiment of the comfort harness of the present disclosure.

FIG. 2 shows a perspective view of one embodiment of the comfort harness for an orthotic brace according to the principles of the present disclosure.

FIG. 3 shows an exploded view of one embodiment of a crescent portion of a comfort harness for an orthotic brace according to the principles of the present disclosure.

FIG. 4A shows a first front perspective view of one embodiment of a crescent portion of a comfort harness for an orthotic brace according to the principles of the present disclosure.

FIG. 4B shows a second front perspective view of one embodiment of a crescent portion of a comfort harness for an orthotic brace according to the principles of the present disclosure.

FIG. 5A shows a first back perspective view of one embodiment of a connector of a comfort harness for an orthotic brace according to the principles of the present disclosure.

FIG. 5B shows a second back perspective view of one embodiment of a connector portion of a comfort harness for an orthotic brace according to the principles of the present disclosure.

FIG. 6 shows a third back perspective view of one embodiment of a connector portion of a comfort harness for an orthotic brace according to the principles of the present disclosure.

### DETAILED DESCRIPTION OF THE DISCLOSURE

There are many forms of orthoses, or devices used externally to modify the structure and/or function of the skeletal and/or neuromuscular systems of the body. For example, there are orthoses that are applied to the neck, to the spine, to the upper limbs, and to the lower limbs. Additionally, there are many different purposes for using orthoses ranging from rehabilitative to prophylactic. Rehabilitation braces are typically used to limit the movement of a portion of the body following an injury or a surgery.

Certain rehabilitation braces, for example orthopedic arm braces, typically immobilize the arm and/or limit the motion in the shoulder, the elbow and/or the wrist. These braces provide a mechanism to reduce the range of motion for a healing limb. The ability to limit flexion and extension are important features for an effective orthopedic shoulder brace, as are limited abduction and adduction. In certain cases, limiting both internal and external rotation is also important. To maximize the benefits of an orthopedic brace it must be properly fitted and adjusted to the patient. Adjustment variables include fitting patients of various sizes and body proportions, and accommodating a variety of possible surgical or injury sites. The adjustment of the brace will also be continual as the patient heals and can tolerate larger ranges of motion, as swelling is reduced, and the like. At times there may also be readjustment of the braces to adapt to accessories and/or product upgrades.

It is understood that wearing an orthotic brace for an extended period of time can be uncomfortable, and can even lead to additional injuries such as abrasions or strain on other parts of the body, such as the neck or back when wearing an arm immobilizing brace. One embodiment of the present disclosure is a comfort harness strap system configured to connect to a multitude of sling designs. In certain embodiments, the comfort harness connects to a front and a portion of a sling. In some cases, the comfort harness covers a multitude of size ranges and has adjustable straps.

The unique adjustability of the current invention allows this brace to be universally fit and easily removed. The detachable closure system interfacing with the crescent shaped pad has two hook and loop removable straps and a front strap that is fixed to a rotation ring with a lower quick connect buckle. The ring feature allows this front strap to rotate naturally on the crescent shaped pad which sits anatomically on the chest and shoulder. The crescent shaped pad with the orthotic brace can be quickly removed and adjusted while the patient is lying in bed or in a seated position. This facilitates easy access for physician assessment or removal of the brace for postoperative procedures.

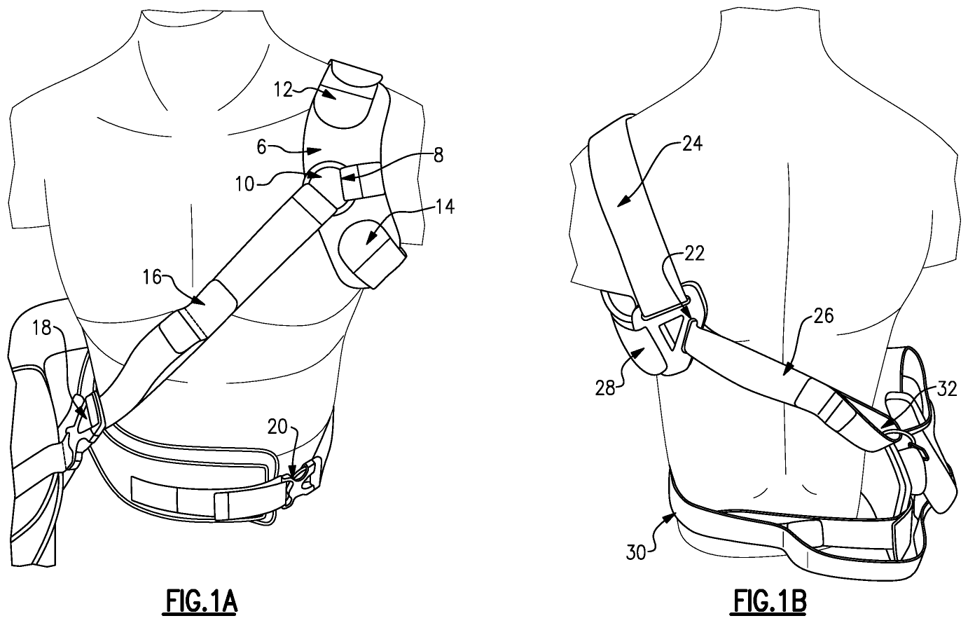

Referring to FIG. 1A, a front perspective view of one embodiment of the comfort harness of the present disclosure is shown. More specifically, one embodiment of the comfort harness of the present disclosure has a crescent shaped pad 6 that cradles the front portion of a wearer's chest/shoulder. In some cases, straps are joined with hook and loop 12, 14 for adjustment over the shoulder and under the arm of the wearer. In some cases, a d-ring 10 is attached 8 to the crescent pad to provide for a cross-body strap 16, which connects to a sling, or other orthotic, via a connector 18. In some cases, the strap 16 is adjustable 16 using hook and loop, or the like, to properly fit a wearer. In some cases, the orthotic, for which the comfort harness is attached, has a closure 20 to secure the orthotic around the waist of a wearer. One benefit of the crescent pad is to prevent the d-ring form touching the wearer and causing abrasions. Another benefit of the crescent pad 6 is to disperse the load across a larger region of chest/shoulder.

Referring to FIG. 1B, a back perspective view of one embodiment of the comfort harness of the present disclosure is shown. More specifically, an upper back strap 24 (which connects to the upper portion of the crescent pad 6 via hook and loop 12) is also adjustable about a multi-strap connector 22. In some cases, the comfort harness has a custom back pad that is a strap multi connector 22 for joining an upper back strap 24, a lower back strap 26 that is cross-body, and an under arm strap 28 that connect to the lower portion of the crescent pad 6 via hook and loop 14. Ion certain embodiments, the lower cross-body back strap 26 connect to a sling, or other orthotic via a connector 32.

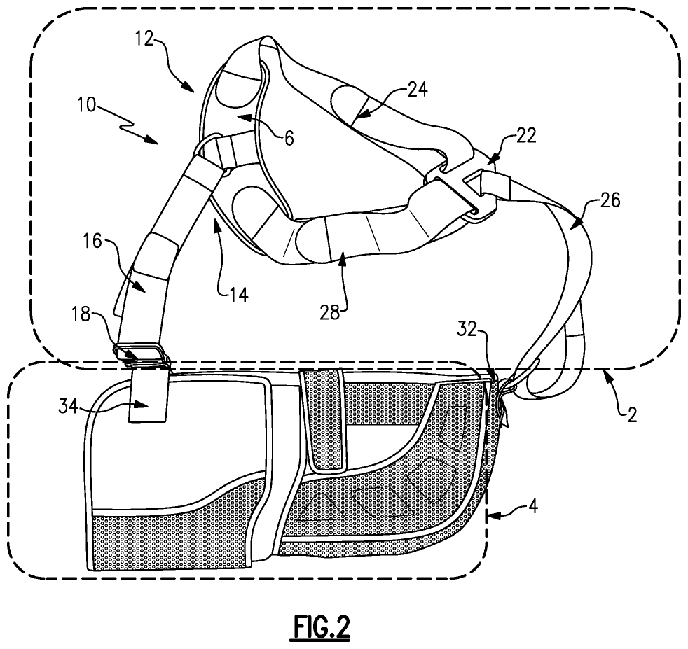

Referring to FIG. 2, a perspective view of one embodiment of the comfort harness for an orthotic brace according to the principles of the present disclosure is shown. More specifically, a sling, or orthotic 4, is used in conjunction with the comfort harness 2 of the present disclosure. On benefit of the comfort harness of the present disclosure is that the straps are easily removable from the front, allowing a wearer to be in a chair or bed and have the straps removed for added comfort. Additionally, the comfort harness design keeps the straps away for the wearer's neck where it could rub of chafe.

Still referring to FIG. 2, certain embodiments of the comfort harness 2 attach to an orthotic via connectors 18 and 32. A crescent pad 6 and a multi-strap connector 22 provide for the attachment of an under arm strap 28, a front cross-body strap 16 an upper back strap 24 and a lower cross-body back strap 26. Each of these strap (16, 24, 26, 28) are adjustable via hook and loop, or the like, for accurately, and comfortably fitting a variety of different sized wearers. In certain embodiments, the cross-body straps 16, 26 are made of non-stretching fabric. In certain embodiments, the under arm 28 and upper back strap 24 straps that connect to the lower and the upper portions of the crescent, respectively, are made of stretchable fabric. In some embodiments, the multi-strap connector 22 is formed of LDPE/foam plastic, or the like.

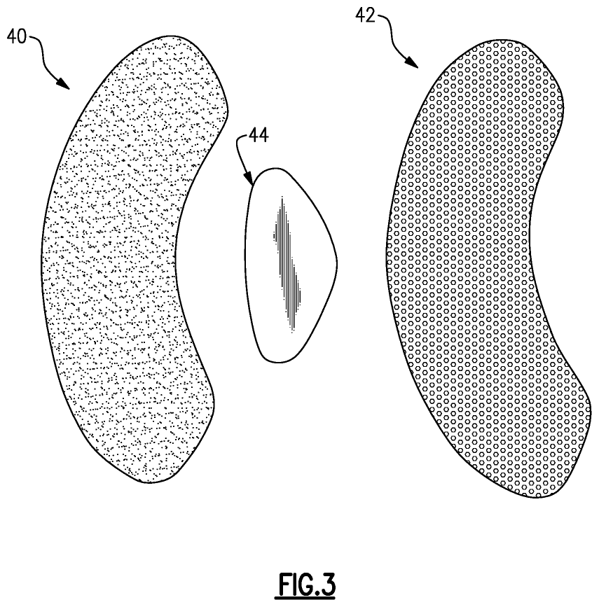

Referring to FIG. 3, an exploded view of one embodiment of a crescent portion of a comfort harness for an orthotic brace according to the principles of the present disclosure is shown. More specifically, in one embodiment a top layer 40 comprises loop laminate and a bottom layer 42 comprises mesh with a cooling fabric, or the like for added comfort against the wearer's body. In some embodiments, a middle layer of LDPE reinforces the crescent to prevent distortion when the harness is under tension.

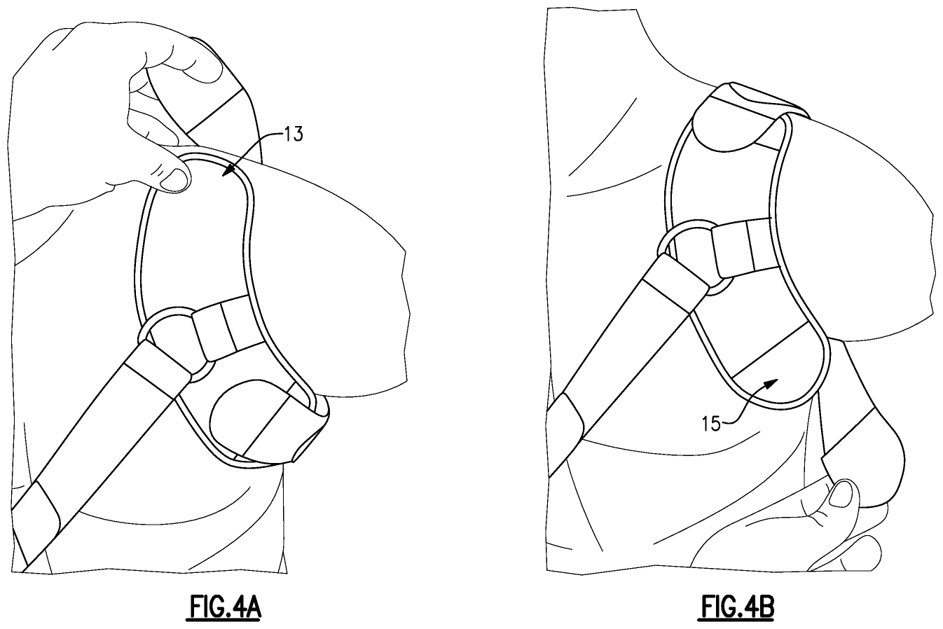

Referring to FIG. 4A, a first front perspective view of one embodiment of a crescent portion of a comfort harness for an orthotic brace according to the principles of the present disclosure is shown. More specifically, the crescent shaped pad is adjustable at a top portion 13. This provides for comfort adjustment and ease of use for a wearer. This is particularly true when the wearer is sitting in a chair or lying down, the harness can be adjusted or even disconnected easily by the wearer.

Referring to FIG. 41, a second front perspective view of one embodiment of a crescent portion of a comfort harness for an orthotic brace according to the principles of the present disclosure is shown. More specifically, the crescent shaped pad is adjustable at a bottom portion 15. This also provides for comfort adjustment and ease of use for a wearer. This is particularly true when the wearer is sitting in a chair or lying down, the harness can be adjusted or even disconnected easily by the wearer

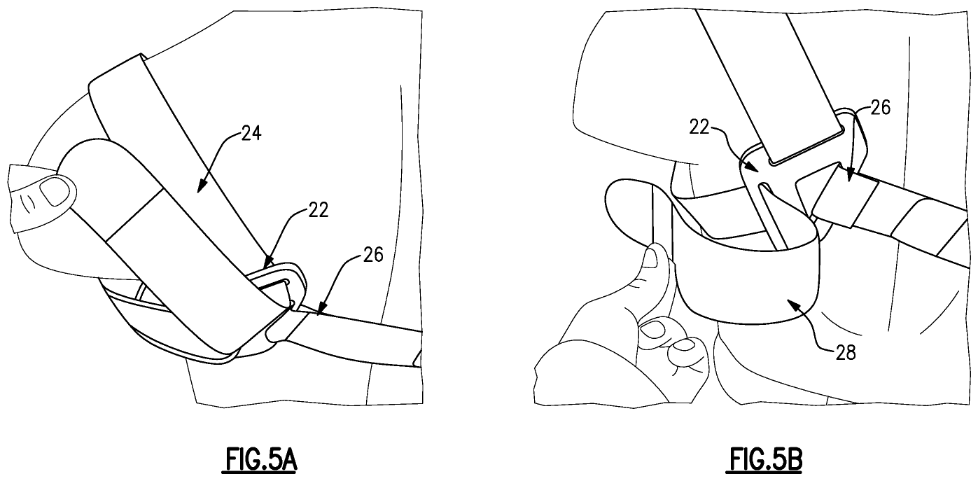

Referring to FIG. 5A, a first back perspective view of one embodiment of a connector of a comfort harness for an orthotic brace according to the principles of the present disclosure is shown. More specifically, an upper back strap 24 is fully adjustable about a multi-strap connector 22 in reference to the other straps, including a back cross-body strap 26. Referring to FIG. 5B, a second back perspective view of one embodiment of a connector portion of a comfort harness for an orthotic brace according to the principles of the present disclosure is shown. More specifically, an under arm strap 28 is fully adjustable in relation to the multi-strap connector 22 in relation to the other straps, including a back cross-body strap 26. One benefit of this embodiment of the comtfort harness is that the multi-strap connector pad in the back of the wearer, when worn, is designed for comfort and low profile fit (e.g., semi-flexible).

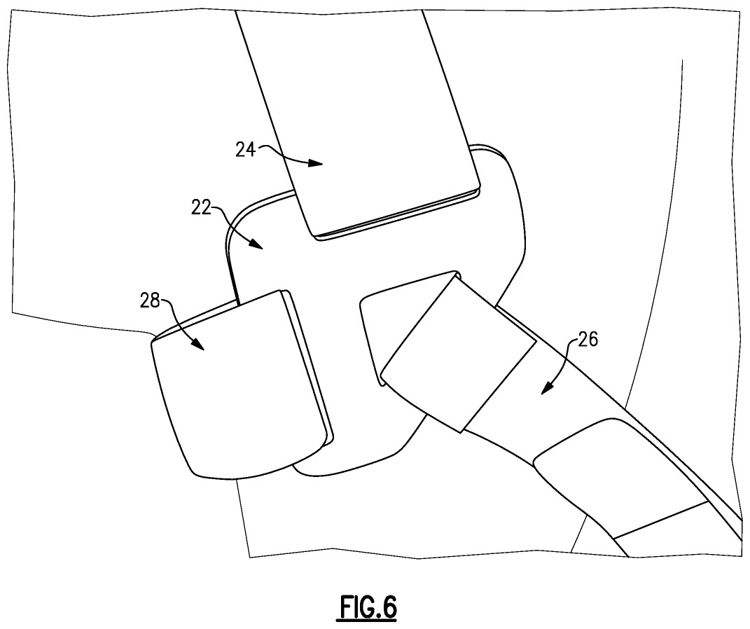

Referring to FIG. 6, a third back perspective view of one embodiment of a connector portion of a comfort harness for an orthotic brace according to the principles of the present disclosure is shown. More specifically, a multi-strap connector 22 is proportioned to accommodate multiple straps. In some cases there are three straps reversibly connected to the connector 22 via hook and loop, or the like. I some embodiments there are three straps 24, 26, 28. In some cases some or all of the straps are constructed of stretchable materials. In some cases one or more of the straps are permanently connected to the multi-strap connector. In some case, the strap material is not stretchable.

While various embodiments of the present invention have been described in detail, it is apparent that various modifications and alterations of those embodiments will occur to and be readily apparent to those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having,” and variations thereof herein, is meant to encompass the items listed thereafter and equivalents thereof as well as additional items while only the terms “consisting of” and “consisting only of” are to be construed in a limitative sense.

The foregoing description of the embodiments of the present disclosure has been presented for the purposes of illustration and description. It is not intended to be exhaustive or to limit the present disclosure to the precise form disclosed. Many modifications and variations are possible in light of this disclosure. It is intended that the scope of the present disclosure be limited not by this detailed description, but rather by the claims appended hereto.

A number of implementations have been described. Nevertheless, it will be understood that various modifications may be made without departing from the scope of the disclosure. Although operations are depicted in the drawings in a particular order, this should not be understood as requiring that such operations be performed in the particular order shown or in sequential order, or that all illustrated operations be performed, to achieve desirable results.

While the principles of the disclosure have been described herein, it is to be understood by those skilled in the art that this description is made only by way of example and not as a limitation as to the scope of the disclosure. Other embodiments are contemplated within the scope of the present disclosure in addition to the exemplary embodiments shown and described herein. Modifications and substitutions by one of ordinary skill in the art are considered to be within the scope of the present disclosure.

What is claimed:

1. A comfort harness for an orthotic, comprising:
a crescent-shaped pad having an upper portion and a lower portion, the upper portion being configured to reversibly attach to an upper back strap and the lower portion being configured to reversibly attach to an under arm strap;
a multi-strap connector being configured to reversibly attach to the upper back strap and the under arm strap; and
at least one cross-body strap being configured to reversibly attach to an orthotic.

2. The comfort harness for an orthotic according to claim 1, wherein the at least one cross-body strap comprises a first and a second cross-body strap.

3. The comfort harness for an orthotic according to claim 2, wherein the first cross-body strap is reversibly attached to the crescent-shaped pad and reversibly attached to the orthotic.

4. The comfort harness for an orthotic according to claim 3, wherein the second cross-body strap is permanently attached to the multi-strap connector and reversibly attached to the orthotic.

5. The comfort harness for an orthotic according to claim 1, wherein the crescent-shaped pad is reinforced.

6. The comfort harness for an orthotic according to claim 1, wherein the crescent-shaped pad further comprises cooling fabric.

7. The comfort harness for an orthotic according to claim 1, wherein the upper back and the under arm straps comprises stretchable fabric.

8. The comfort harness for an orthotic according to claim 1, wherein the multi-strap connector comprises two or more slots for receiving and orienting straps.

9. The comfort harness for an orthotic according to claim 8, wherein the multi-strap connector comprises a LDPE foam laminate.

10. An adjustable comfort harness for an orthotic, comprising:
a crescent-shaped pad having an upper portion and a lower portion, the upper portion being configured to reversibly attach to an upper back strap and the lower portion being configured to reversibly attach to an under arm strap;
a multi-strap connector comprising at least two slots for receiving straps, the multi-strap connector being configured to reversibly attach to the upper back strap and the under arm strap; and
a first and a second cross-body strap being configured to reversibly attach to an orthotic.
